(19) United States
(12) Patent Application Publication
(10) Pub. No.: US 2007/0016283 A1
(43) Pub. Date: Jan. 18, 2007

(54) Micro-thin film structures for cardiovascular indications

(75) Inventors: Skott E. Greenhalgh, Glenside, PA (US); John-Paul Romano, Chalfont, PA (US)

Correspondence Address: INSKEEP INTELLECTUAL PROPERTY GROUP, INC, 2281 W. 190TH STREET, SUITE 200, TORRANCE, CA, 90504, US

(73) Assignee: Stout Medical Group, Inc.

(21) Appl. No.: 11/478,403

(22) Filed: Jun. 28, 2006

Related U.S. Application Data
(60) Provisional application No. 60/696,018, filed on Jun. 28, 2005.
(60) Provisional application No. 60/762,338, filed on Jan. 25, 2006.

(51) Int. Cl. A61F 2/06

(52) U.S. Cl. 623/1.15

(57) ABSTRACT

A thin lining for use within a body lumen having a variety of uses and particularly suited for protecting the intimal lining of a vessel, thereby preventing the release of embolic materials through the intima and into the bloodstream, either naturally or during the implantation of a prosthetic device. The thin lining is porous enough to promote ingrowth and thin enough to avoid migration with minimal radial force being placed on the vessel walls, making the lining also suited for applications such as blocking the entrance to an aneurysm.

### CROSS-REFERENCE TO RELATED DOCUMENTS

This application is related to and claims priority benefit of U.S. Provisional Patent Application Ser. No. 60/696,018, filed Jun. 28, 2005, entitled Micro-Thin Film Structures For Cardiovascular Indications; and U.S. Provisional Patent Application Ser. No., 60/762,338, filed Jan. 25, 2006, entitled Micro-Thin Film Structures For Cardiovascular Indications II. These applications are also hereby incorporated by reference herein.

### BACKGROUND OF THE INVENTION

Vulnerable plaque can be loosely described as a collection of fat and proteins (and sometimes mineral or necrotic matter) in an arterial wall, covered by the intima, a thin layer of tissue. The intima is susceptible to tearing, which could spill the fat and protein contents. If this occurs, platelets and other coagulation cascade participants stick to the area, and can form dangerous vessel-occluding clots.

In comparison to the soft tissue of an arterial wall, most stents are strong, rigid, and have many edges that could tear the intima during or after implantation. Though much attention has been focused on stent designs, comparatively little innovation has been directed toward developing a prosthetic arterial liner or a protective barrier that would prevent the intima from being torn during stent implantation. Additionally, stents place pressure on the arterial wall, which can lead to intimal hyperplasia. The pressure results not only from the strong outward pressure a stent is designed to exert on a vessel wall, but also from the relative inflexibility of a stent.

There is a need for an implantable device that conforms to the interior of a vessel.

There is further a need for an implantable intravascular device that lines a vessel without exerting unnecessary force on the intima.

### SUMMARY OF THE INVENTION

The present invention is directed to overcome some or all of the aforementioned problems.

One aspect of the present invention provides a prosthetic lining that promotes in-growth resulting in the development of a new endothelial layer. More specifically, a prosthetic lining dimensioned to create and support a microenvironment is provided.

Another aspect of the present invention provides a liner used to cover calcifications.

Yet another aspect of the present invention provides a device that covers vulnerable plaque and prevents ruptures.

Still another aspect of the present invention provides a device that covers side branches, tumor feeding vessels, or aneurysm necks.

A further aspect of the present invention uses pure ethanol to spin polymers while maintaining highly clean, non-toxic membranes.

One aspect of the present invention uses a sub-micron level nanoskin for use as a cover over the foil prosthetic device.

It is also an aspect of the present invention to use a nanoskin in a chemically ruptured form to coat sub-30 micron sized stent struts or wires while allowing the wires to move freely without changing the flexibility, porosity or radial force of the stent.

Electrospinning methods are disclosed in U.S. application Ser. No. 10/856,893, entitled Filament Based Prosthesis, filed May 27, 2004, U.S. application Ser. No. 10/314,086, entitled Covering And Method Using Electrospinning Of Very Small Fibers, filed Dec. 6, 2002, U.S. application Ser. No. 10/313,161 entitled Stent Having Electrospun Covering And Method, filed Dec. 6, 2002, U.S. application Ser. No. 10/313,496 entitled, Electrospun Skin Capable of Controlling Drug Release Rates and Method, filed Dec. 6, 2002, and U.S. application Ser. No. 10/313,835 entitled Coated Stent And Method For Coating By Treating An Electrospun Covering With Heat Or Chemicals filed Dec. 6, 2002 all of which are hereby incorporated by reference.

The present invention relates to multi-functional, thin-walled tubular structure. The device may be used as a stent or similar supporting structure, as a prosthetic intima, re-lining a vessel wall. The prosthetic liner may be porous, thereby promoting in-growth such that a new endothelial lining will form over the surface of the device. Alternatively, the material used may have a very low porosity, such as a foil, thereby relying on the many gaps between the low-porosity struts to allow in-growth.

In addition to protecting the delicate intima of a vessel, various embodiments of the present invention present advantageous uses for treating other vascular indications such as calcified lesions, aneurysms, clots, weakened vascular walls, shunts, and the like. More generally, the various embodiments of the present invention are particularly suited for use in situations where it is desirable to protect tissue or matter lining the vascular walls, either from future harm from natural erosive elements or forces brought about by the blood stream pressures and velocities, or from additional prosthetic implants to be placed at a particular vascular site. The various embodiments of the present invention are also suited to protect the body from emboli dislodged from the vessel walls, either naturally or during the implantation of a prosthetic. The various embodiments of the present invention are additionally ideal for creating scaffold for progenitor cells from the blood stream and/or endothelial cells to heal into via in-growth.

Preferably, the device is formed from a tube of foil that is ¼^{th} to 1/20^{th} the thickness of typical coronary stent material. The device includes a plurality of members or struts that are ¼^{th}- 1/20^{th} the width and length of typical stent struts. Correspondingly, the number of struts per unit length and/or perimeter is two to ten times that of a conventional stent.

Other characteristics of the present invention that distinguish it from typical stents include:

1) Extremely thin walls (0.00015″-0.002″ vs. 0.006″-0.010″ typical stents)

2) Component structure (rings, ribbons, etc.) facilitates bending around curves

3) Deployment foreshortening reduces length (by definition) and reduces porosity

These attributes encourage adherence to the vessel wall without imposing significant radial forces. For example, the extremely thin walls not only promote in-growth, which anchors the prosthetic to the walls, they take advantage of the hydrodynamic boundary layer phenomenon present whenever a fluid flows over or through a stationary object, such as when blood flows through a vessel. The boundary layer arises from the fact that the fluid at the very surface of a stationary object is also stationary. The boundary layer in a blood vessel is thus a transitional layer of blood between the blood that is considered the blood stream and the vessel itself, including the thin, stationary film at the very surface of the vessel. Because the fluid velocities in the boundary layer are much slower than those in the blood stream, the amount of shear stress placed on the thin-walled prosthetic is very small, thereby significantly reducing the radial force necessary to prevent migration after implantation but before in-growth has occurred. In addition to being thin, the walls of the prosthetic are smooth and streamlined, typically being etched from flat foil materials. Hence, the fluid forces encountered by the prosthetic are further reduced.

One embodiment of the present invention provides a foil stent that incorporates a spiral design, which creates a very flexible device when unexpanded and expanded. The spiral design includes a plurality of helical ribbons integrated to form a cylindrical stent-like tubular device. The ribbons may be completely independent or include any number of connection points where the ribbons are connected together.

The invention is inserted percutaneously into a vessel (or other conduit) in the body. Typical stents exhibit radial force sufficient to alter the target vessel's radial cross-sectional geometry, at least to some degree. By definition, a “stent” opens or maintains an opening. However, the foil stent disclosed here is intended to conform to the natural radial and longitudinal undulations of a vessel. The device is intended to “blanket” the inside surface of the vessel and follow the natural vessel dilations, contractions, and geometry changes.

### BRIEF DESCRIPTION OF THE DRAWINGS

FIG. 1 is a perspective view of a helical independent embodiment of the present invention;

FIGS. 2-5 are various plan views of embodiments of ribbon/ring designs of the present invention;

FIG. 6 is a perspective view of a ringed embodiment of the present invention;

FIG. 7 is a side elevation of a helical joined embodiment of the present invention;

FIG. 8 is a perspective view of a layered embodiment of the present invention;

FIG. 9 is a perspective view of an covering embodiment of the present invention;

FIG. 10 is a perspective view of a lining embodiment of the present invention;

FIG. 11 is a side elevation of an embodiment of the present invention; and,

FIG. 12 is a side elevation of an embodiment of the present invention.

### DETAILED DESCRIPTION OF THE INVENTION

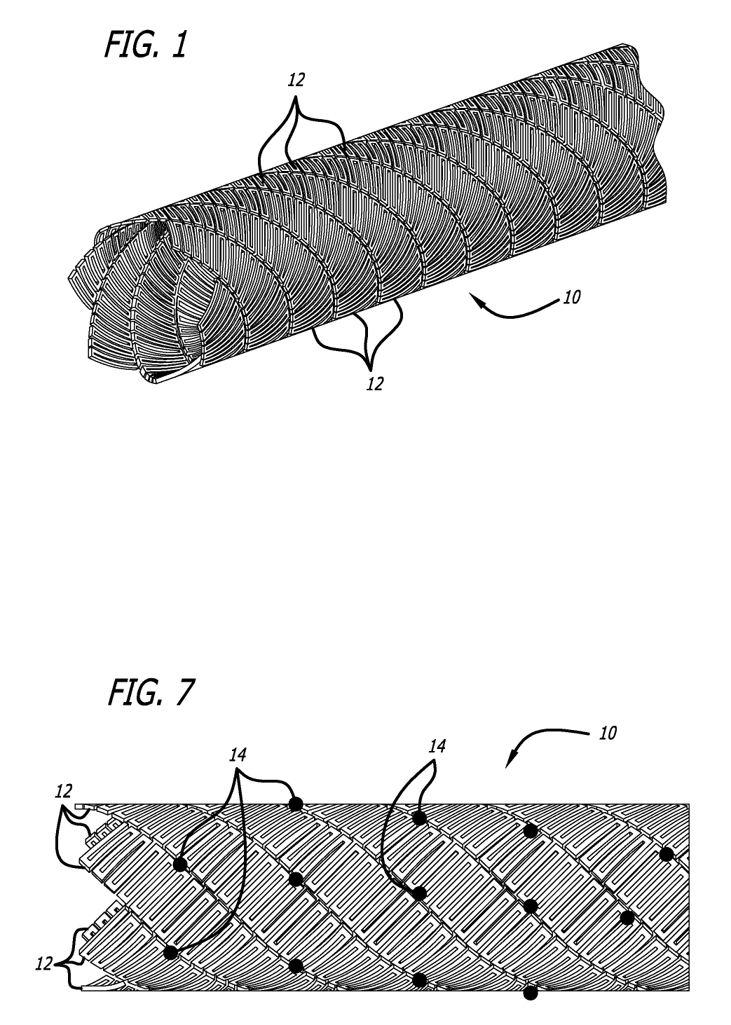

Referring now to the figures and first to FIG. 1, there is shown one embodiment of a device 10 of the present invention. The device 10 is a stent-like device made of a very thin, foil-like material. Like the device 10 in FIG. 1, the devices of the present invention generally comprise a plurality of ribbons 12. In the embodiment of FIG. 1, there are six ribbons 12 arranged to form the cylindrical device 10. The ribbon design of the present invention provides a wide range of design possibilities. Hence, the description of the present invention will be divided into ribbon designs, materials, construction, and uses. It will be understood that the various embodiments discussed under each of these headings can be used in any combination with each other.

Ribbon Design

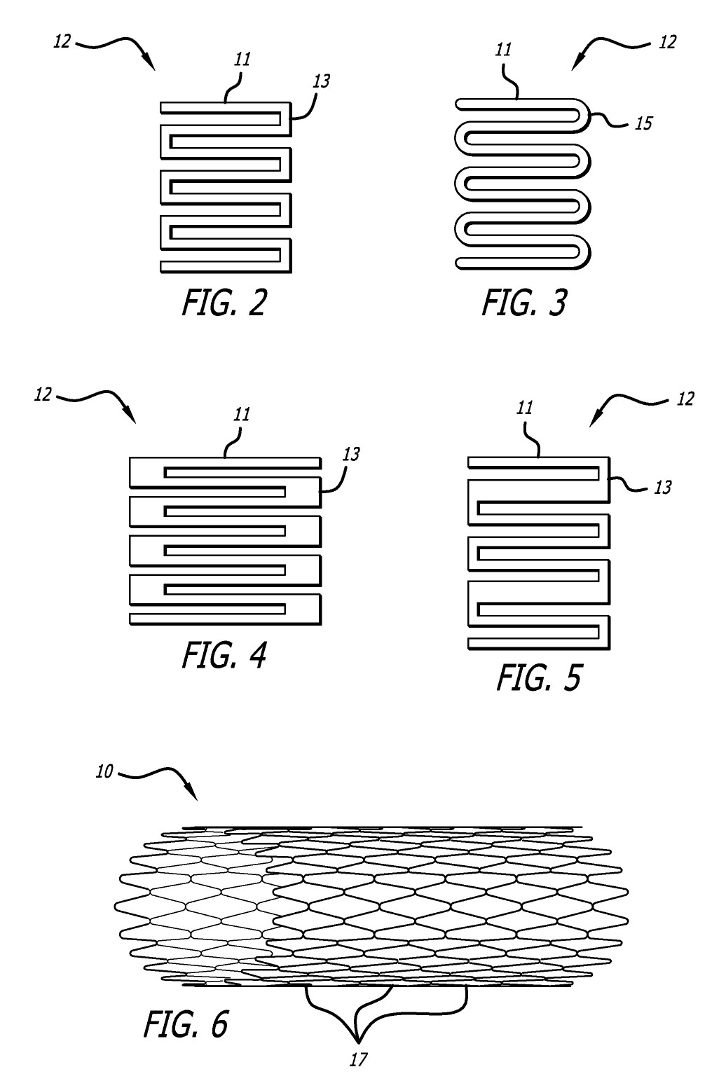

Referring again to FIG. 1, there is shown a device 10 comprised of a plurality of ribbons 12. Each of these ribbons 12 is formed of a thin foil-like material that follows a serpentine pattern. The serpentine pattern is more clearly shown in FIG. 2 as being formed of a plurality of long struts 11 joined at right angles by shorter struts 13. Another serpentine pattern, shown in FIG. 3, includes long struts 11 joined by curved struts 15 to form a more sinusoidal pattern. The ribbon variations shown in FIGS. 2 and 3 are provided to show that a wide variety of shapes are possible. Strut length, width and orientation are selected based on parameters such as flexibility, radial strength, expansion, elongation, and surface area.

Additionally, though FIGS. 2 and 3 show each section as having approximately equal widths, as shown in FIG. 4, the width of the shorter struts 13 may differ from those of the longer struts 11. Further, as shown in FIG. 5, the width of selected sections may be varied to provide desired characteristics regarding the aforementioned parameters, particularly, radial strength, flexibility, and surface area. Similarly, the porosity, or spaces between the sections, may also be uniform or varied. Thus, devices having homogenous strut sizes and porosity, and non-homogeneous strut sizes and porosity are considered embodiments of the present invention.

Variations in the arrangement of the struts that make up each ribbon are combined with variations in the shape of the ribbon itself to create the unique performance characteristics of each device 10 of the present invention. For example, the embodiment of the device 10 shown in FIG. 1 is comprised of six helical ribbons 12 juxtaposed to form a generally cylindrical device 10. By varying the pitch of each ribbon 12, more or fewer ribbons are required to form the cylindrical device 10. For example, if the pitch were increased significantly, a single ribbon 12 could be used to form the device 10. A device having fewer ribbons 12, and necessarily greater pitch, will expand more easily and encounter less shortening lengthwise.

Rather than providing helical ribbons 12, FIG. 6 shows a device 10 of the present invention comprised of a plurality of rings 17, connected together longitudinally. This design is similar to more traditional stent designs except that the rings 17 are extremely thin and their patterns are miniaturized compared to corresponding stent designs. Notably, the ribbon designs shown in FIGS. 2-5 may also be used in rings 17. Moreover, any pattern, such as those presently used in stents or otherwise, may be incorporated into the ring/ribbon design of the present invention. Examples of existing stent designs that may be miniaturized and formed into a foil device 10 of the present invention include, buy are by no means limited to, Palmaz designs, multilink designs such as those by Guidant, Inc., the S7 design by Medtronic, Inc., the NIR design by Boston Scientific, Inc., the Cypher design by Johnson and Johnson, Inc., etc. Examples of other designs include, but are by no means limited to, zigzags, connected circles, grids, diamonds, squares, fishnet, octagonal, honeycomb, to name a few.

The ribbons 12 and rings 17 may be manufactured in a variety of ways. An example of on manufacturing technique is to laser cut the pattern from a tube having a desired diameter and thickness. Another acceptable technique is to electro-chemically machine or etch a flat sheet, then roll and weld the sheet into a tube. Yet another example of a manufacturing technique used to make the device 10 is to form a fine-wire pattern and use metal deposition on the pattern to build the device 10.

The design of the ribbons 12 and rings 17 greatly impact the flexibility of the device 10. For example, utilizing helixes, such as those shown in FIG. 1, oriented at approximately 45 degrees to the longitudinal axis of the device provides a greater degree of flexibility than a flatter helix. Reducing the number of connection points 14 between ribbons also improves flexibility. A device 10 having a plurality of ribbons 12 or rings 17 that are connected together with few connection points 14 results in a device that bends easily around a curve because the ribbons 12 or rings 17 tend to separate from each other along the outside radius of the curve and overlap around the inside of the curve. Hence, flexibility is greatly improved over designs where overlap is not possible.

Materials

A variety of materials may be used to form the ribbons 12 and rings 17. Examples of metallic materials include stainless steel, NiTi, CoCr, tantalum, titanium or any other material having desirable properties. The metals may also be impregnated with a drug. Alternatively, the ribbons 12 and rings 17 could be polymeric. Regardless of the materials used, one or more of the ribbons 12 and rings 17 may be coated with a drug or agent.

One embodiment of a polymer ribbon 12 or ring 17 is formed using the techniques shown and described in U.S. application Ser. No. 10/313,496 entitled, Electrospun Skin Capable of Controlling Drug Release Rates and Method, the entirety of which is incorporated by reference herein. For example, pure ethanol may be spun into a polymeric sheet using the technique of this application. The result would be a very thin, completely sterile sheet of material, which could then be stamped, cut, or otherwise shaped and rolled into ribbons 12 or rings 17. The ethanol could also be impregnated with a drug, such as NO or paclitaxel, if it is desired to deliver a medicament to the target site.

Texturing may also be used to provide varying characteristics to the device. For example, the exterior of the device may be textured to increase the grip the device has on the interior of the vessel. Texturing may also promote in-growth. The interior of the device may also be textured, to promote the formation of a thin endothelial layer, or remain smooth to reduce flow resistance therethrough.

Alternatively, the device 10 may be formed and then coated with a polymer. For example, U.S. application Ser. No. 10/313,835 entitled Coated Stent And Method For Coating By Treating An Electrospun Covering With Heat Or Chemicals, the entirety of which is incorporated by reference herein, provides a coating method that may be employed to coat the device 10 with a polymer without filling the interstices between the sinusoidal elements. Thus, the polymeric coating would not affect the flexibility of the device 10. Nor would the coating flake off during expansion. The device 10 could be alternatively, or additionally, covered with a metal micromesh textile and welded as specific locations (not shown).

Device Construction

As with the other aspects of the device 10 of the present invention, the manner in which the device is constructed, using the various ribbons 12 and rings 17, greatly affects the performance of the device. The versatility of the present invention is demonstrated by the various construction configurations.

One embodiment of the present invention, shown in FIG. 1, provides six ribbons 12 that are juxtaposed, yet independent. This configuration allows the various ribbons 12 to shift relative to each other, giving the resulting device 10 maximum flexibility and conformity.

Altering the device 10 slightly provides varying degrees of flexibility. For example, FIG. 7 shows a device 10 having a plurality of ribbons 12 that are joined together at spaced apart connection points 14. Increasing the number of connection points 14 reduces the flexibility and increases the strength of the device 10. Additionally, varying the location of the connection points, rather than the number of connection points, also allows variations in flexibility.

The device 10 may be constructed to be expandable or self-expanding. One embodiment provides an expandable device 10 formed to a delivery diameter such that, in a delivery catheter, the device 10 is in a relaxed state. Upon delivery, a balloon or similar mechanism is used to expand the device 10 to a diameter sufficiently large enough to hold the device 10 against an interior wall of the targeted vessel.

Regarding the self-expanding embodiment, the device 10 is formed to a deployed diameter that is at least as large as the targeted vessel. The device is then compressed and inserted into a delivery catheter. Upon delivery, the device 10 exits the delivery catheter and expands against the inner wall of the targeted vessel.

If additional radial force is desired, such as to open a stenosed or prolapsed vessel, a backbone (not shown) may be incorporated into the device 10. Alternatively, one or more of the ribbons 12 may be formed using thicker sinusoidal members.

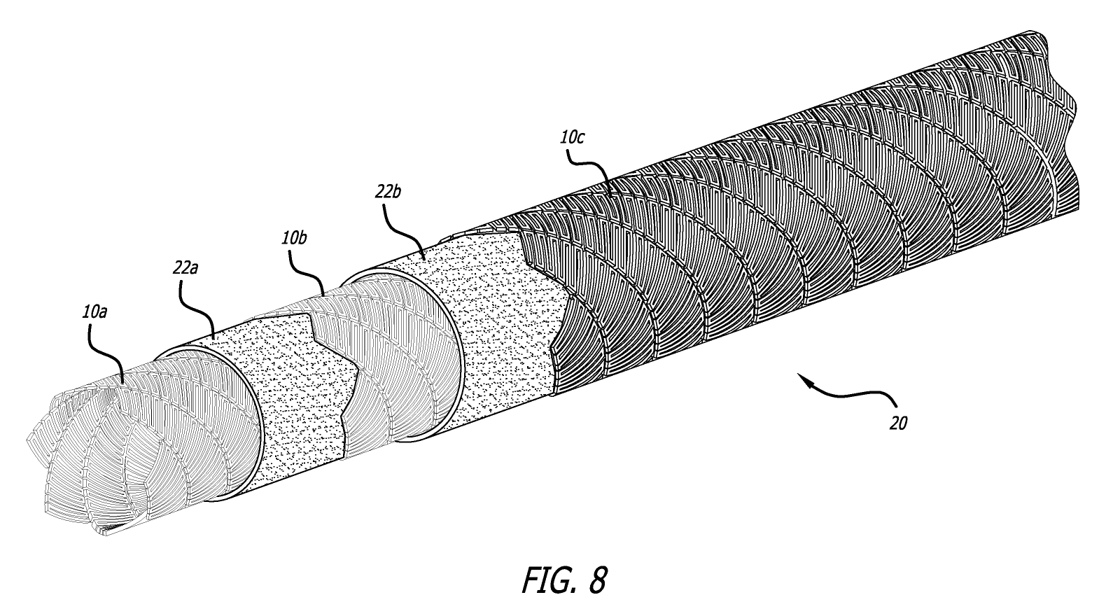

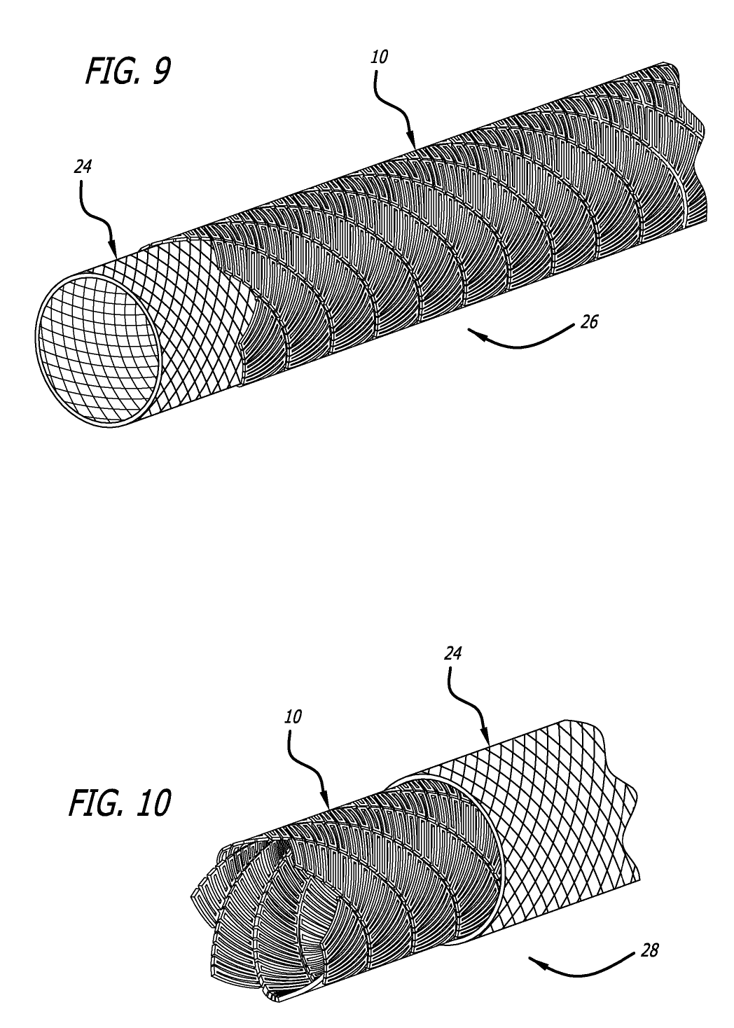

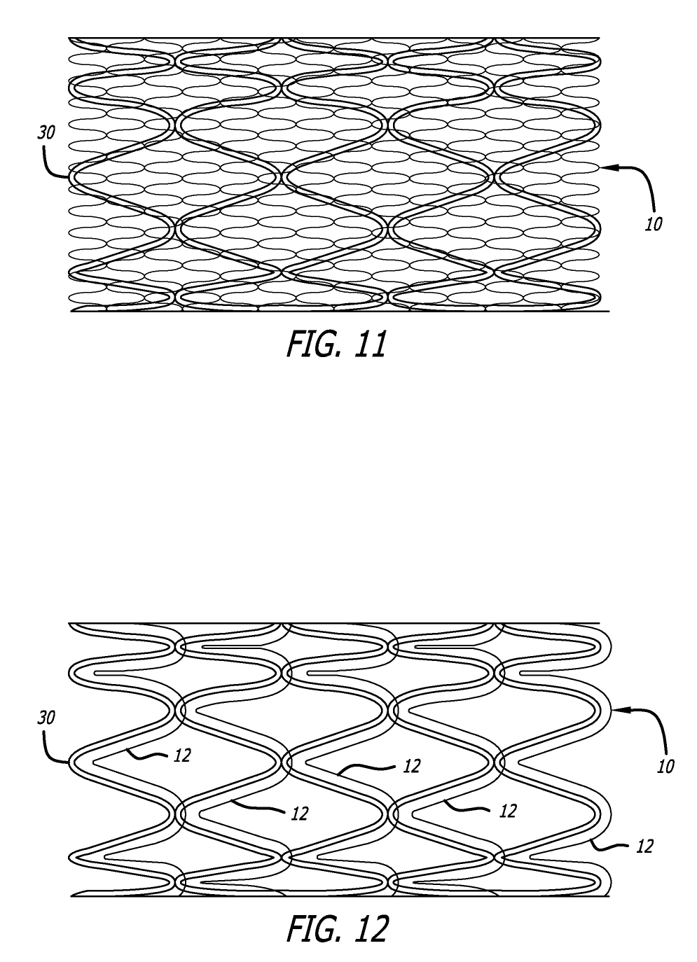

Turning now to FIG. 8, it is shown how the device 10 may be used to form a multi-layer structure 20. The structure 20 includes three layers of the device 10, specifically, layers 10a, 10b, and 10c. Between the layers are optionally positioned two layers of polymer 22a and 22b. One skilled in the art will realize that the layers 10a, 10b and 10c may be formed from identical material or may have differing characteristics such as strut length, wall thickness, strut placement, strut design, pitch, material, and the like. The use of multiple layers (three are shown but two or more than three may also be used) allows structural strength to be developed using very thin materials. The use of multiple layers forms a three dimensional architecture, with increased surface area, that is ideal for cell migration and healing. The increased surface area also acts as a healing, bioactive surface that recruits cells (progenitor cells, stem cells, etc.) from the native vessel wall or from the blood itself. Hence, a living stent is formed. Preferably, each layer 10a-10c is on the order of 10-30 microns thick, though other thickness may be used as application dictates. The layered devices 10a-10c also provide additional surfaces onto which agent coverings or coatings may be applied.

The optional polymer layers 22a and 22b shown in FIG. 3 are shown as non-braided fibrous layers, such as those produced via electrospinning, but may be woven, braided, knitted, pressed, rolled, or the like. Additionally, these polymer layers may be degradable, non-degradable, or a combination thereof. The polymer layers 22a and 22b provide impregnable vehicles for drug delivery. Additionally, due to the sandwich-like architecture of the structure 20, it is possible to “pack” more polymer in between the layers 10a, 10b, and 10c than would otherwise be possible if a prosthetic were simply wrapped or lined with a polymer.

Uses

The device 10 of the present invention may be used as a stand-alone prosthetic, providing light structure or a lining such as a prosthetic intima, or as a blockade that prevents fluid from entering a side branch or aneurysm in a body lumen. For example, the device 10 could be placed in a blood vessel so that it covers the opening of an aneurysm. As ingrowth occurs, the device 10 will completely block the opening of the aneurysm.

Another aspect of the present invention incorporates the device 10 as a liner or a covering for a prosthetic such as a stent. Turning to FIGS. 4 and 5, FIG. 4 shows the device 10 wrapped around a stent 24 to form a covered prosthetic 26. Rather than implanting the device 10 into the receiving vessel prior to implanting a stent, as discussed above, the prosthetic 26 is constructed with a stent 24 joined or concentrically nested with the device 10 prior to implantation. Similarly, FIG. 5 shows a prosthetic 28 constructed of a stent 24 lined with the device 10, the device 10 being either concentrically nested or joined with the stent 24.

In making prosthetics 26 and/or 28, the stents 24 may be joined with the devices 10 using a variety of techniques and materials including, but not limited to, glue, welds, sutures, crimping, tethers, and the like. If concentrically nested, a friction or interference fit may be employed. Additionally, though the stents 24 are shown as wire braided stents, any stent form may be lined or covered with the device 10 of the present invention. The stents 24 may be self-expanding or expandable as the design of device 10 remains conforming during expansion.

The covered and lined prosthetics 26 and 28 are advantageous over conventional stents as the device 10 provides increased surface area for delivering bioactive substances. Additionally, the device 10 of the covered prosthetic 26 is usable to control elution rates if the stent 10 is a drug coated or covered stent. Additionally, if a heavy-walled orthopedic stent is used as stent 10 in prosthetic 26 or 28, the device 10 is usable to control where fillings (BMP's, collagens, bone chips, cement (PMMA, ceramics), ceramic particles, etc.) are to be administered.

It is also an embodiment of the present invention to use a foil covering or liner 10 in combination with a prior art stent. For example, FIG. 11 shows a covering 10 over a stent 30 having a design similar to an S7 stent, made by Medtronic, Inc. The device 10 has the same design as the stent 30, only on a much smaller scale. This device 10 could also be used as a liner for the stent 30, or both. Moreover, as discussed above, the liner or cover can be made by miniaturizing any existing stent design. Examples of existing stent designs that may be miniaturized and formed into a foil device 10 of the present invention include, buy are by no means limited to, Palmaz designs, multilink designs such as those by Guidant, Inc., the S7 design by Medtronic, Inc., the NIR design by Boston Scientific, Inc., the Cypher design by Johnson and Johnson, Inc., etc. Examples of other designs include, but are by no means limited to, zigzags, connected circles, grids, diamonds, squares, fishnet, octagonal, honeycomb, to name a few.

FIG. 12 shows a covering 10 over a stent 30 having a design similar to an S7 stent, made by Medtronic, Inc. Unlike the embodiment of FIG. 11, however, the covering 10 of FIG. 12 has approximately the same scale and dimensions as the stent 30 it is covering.

### EXAMPLE

Favorable results have been achieved forming a device 10 according to the present invention with the following characteristics:

OD: 0.075″

Wall thickness: 0.00075″

Number of ribbons: 6

Gaps: 0.0015″

Element widths: 0.002″ (“Element” refers to 1 complete sinusiodal pattern, peak-to-peak)

Element length: 0.026″

Other Variations

The design of the device 10 lends itself to many variations in addition to those already discussed. One skilled in the art will recognized that many desired characteristics may be achieved by varying the following:

Number of ribbons

Helix angle or pitch

Sinusoidal amplitude

Sinusoidal wavelength

Element size (uniform or mixed sizes)

Longitudinal diameter changes

Longitudinally varying wall thickness

The elements could have varying thicknesses. For example, rather than being flat, the elements could be tapered to provide extremely thin edges that are resistant to crack propagation.

The incorporation of radiopaque markers to assist in precision placement. For example, it is envisioned to use a laser to cut in small holes at the ends and/or through the length of the device to be filled with gold, platinum and their alloys in order to create a radiopacity usable to illuminate the device location. Holes may be from 0.002-0.006″ in diameter, and in any shape.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A prosthetic liner comprising at least one elongate ribbon configured to form a first tubular structure, the ribbon being formed of a thin, narrow strand shaped to create the elongate ribbon such that the elongate ribbon has a width wider than a width of the thin, narrow strand, said elongate ribbon having a length that is longer than a length of the prosthetic liner.

2. The prosthetic liner of claim 1 wherein said thin, narrow strand comprises a narrow strand having a thickness of between 0.00015 inches and 0.002 inches.

3. The prosthetic liner of claim 1 wherein said thin, narrow strand is shaped to form a serpentine pattern.

4. The prosthetic liner of claim 1 wherein said thin, narrow strand is shaped to form a plurality of long struts joined at angles by short struts that are shorter than the long struts.

5. The prosthetic liner of claim 4 wherein said short struts are wider than said long struts.

6. The prosthetic liner of claim 4 wherein said short struts are narrower than said long struts.

7. The prosthetic liner of claim 3 wherein said serpentine pattern comprises a sinusoidal pattern.

8. The prosthetic liner of claim 1 wherein said at least one ribbon comprises a plurality of ribbons helically arranged to form said tubular structure.

9. The prosthetic liner of claim 1 further comprising a second tubular structure concentrically contained within the first tubular structure and a material contained between the first and second tubular structures.

10. The prosthetic liner of claim 9 wherein said second tubular structure comprises at least one elongate ribbon configured to form said second tubular structure, the ribbon being formed of a thin, narrow strand shaped to create the elongate ribbon such that the elongate ribbon has a width wider than a sidth of the thin, narrow strand, said elongate ribbon having a length that is longer than a length of the prosthetic liner.

11. A prosthetic liner comprising:
a plurality of rings, each of said rings being formed of a material having a thickness of between 0.00015 inches and 0.002 inches;
wherein said rings are arranged concentrically and connected together to form a tubular structure;
wherein the concentric rings are arranged such that, when the tubular structure is straight, the rings do not overlap each other.

12. The prosthetic liner of claim 11 wherein each of said rings comprises a thin, narrow strand formed in a serpentine pattern.

13. The prosthetic liner of claim 11 wherein the concentric rings are arranged such that, when the tubular structure is bent to form a curve, the rings overlap each other on the inside of the curve, and are spaced apart on the outside of the curve.

14. A method of protecting the intima of a vascular site comprising:
lining the intima with a thin tubular structure thin enough to reside within a boundary layer the blood stream, thereby preventing migration of the thin tubular structure while obviating a need for placing significant radial force on the intima;
promoting ingrowth of tissue into the thin tubular structure.

15. The method of claim 14 wherein lining the intima with a thin tubular structure comprises lining the intima with a thin tubular structure having a thickness of between 0.00015 inches and 0.002 inches.

16. The method of claim 14 wherein lining the intima with a thin tubular structure comprises lining the intima with a metal foil structure.

17. A method of preventing fluid from entering a luminal aneurysm comprising:
lining a lumen with a thin tubular structure thin enough to reside within a boundary layer the blood stream, thereby preventing migration of the thin tubular structure while obviating a need for placing significant radial force on the intima, said thin tubular structure being positioned to cover an opening of an aneurysm;
promoting ingrowth of tissue into the thin tubular structure.

18. The method of claim 17 wherein lining a lumen with a thin tubular structure comprises lining a lumen with a thin tubular structure having a thickness of between 0.00015 inches and 0.002 inches.

19. The method of claim 17 wherein lining a lumen with a thin tubular structure comprises lining a lumen with a metal foil structure.

20. The method of claim 17 wherein promoting ingrowth of tissue into the thin tubular structure comprises providing a plurality of spaces defined within the thin tubular structure into which tissue may grow.
